(19) United States
(12) Patent Application Publication
(10) Pub. No.: US 2025/0302031 A1
(43) Pub. Date: Oct. 2, 2025

(54) System for Pest Deterrence Utilizing Ozone

(71) Applicant: Purfresh Clean Inc., Hayward, CA (US)

(72) Inventors: Christian DeBlasio, Belmont, CA (US); Grant Still Shatto, III, Castro Valley, CA (US)

(21) Appl. No.: 18/620,893

(22) Filed: Mar. 28, 2024

(51) Int. Cl. A01M 29/12; A61L 9/12

(52) U.S. Cl. CPC A01M 29/12; A61L 9/122; A61L 2209/111; A61L 2209/212

(57) ABSTRACT

An exemplary method for pest deterrence in an interior volume may include moving a portable pest deterrent device into the interior volume; verifying that the interior volume is unoccupied; actuating the portable pest deterrent device to deliver ozonated air to the interior volume; equilibrating ozone concentration in the interior volume within a selected ozone concentration range for an exposure duration; detecting a completion condition; actuating the portable pest deterrent device to cease delivering ozonated air to the interior volume; and waiting, for an ozone clearance duration, for ozone decomposition.

### FIELD OF THE INVENTION

This invention relates to pest control, and more particularly to the use of a portable system for treating an interior volume with ozone for pest control.

### BACKGROUND

There is a broad need for pest control across a wide variety of situations, users, and industries. Pests include insects, birds, rodents and other mammals, and other organisms that desire to consume food items meant for human consumption. In particular, restaurants, breweries, brewpubs, craft brewery taprooms, vineyards, and tasting rooms have particular needs for keeping rodents and insects out of their interior spaces. Rodents and insects enjoy eating grains used for making beer, and grapes used for making wine. If they are allowed to burrow into storage areas for grain and grapes, they can contaminate a quantity of those ingredients far larger than they consume, requiring those ingredients to be discarded at a painful economic cost. Even if the storage areas for food items are clean and clear of vermin, if a person sees a rodent or insects in a public space such as a restaurant, automatically that person will perceive that public space as unclean, and will likely avoid it in the future.

It is known in the art to utilize ozone in a grain silo or grain storage area to control pests in the grain. However, those systems are closed systems, where the ozone is retained within the grain silo or grain storage area. Additionally, such systems are fixed in place relative to the grain silo or grain storage area. Other fixed ozone pest control systems are known in the art as well, such as greenhouse pest control systems using fixed systems are known in the art as well. Those fixed systems require fixed pipes/ducts, fixed valves, and fixed control systems, among other elements. Because they are fixed in place in a structure, such systems are expensive to install and may be bulky. Maintenance on such fixed systems can be difficult and expensive, such as when pipes or ducts located at a height or within a wall must be cleaned or replaced. Further, fixed ozone pest control systems can be used only in the part of a structure to which they are permanently associated. If pest control is required in another part of the structure, those fixed systems cannot perform pest control in that location.

Additionally, known pest control systems that utilize ozone typically apply ozone at such a high level that the ozone kills insects and rodents in the treated air volume. Dead rodents may expire in an attic, a basement, inside walls, or in other spaces not readily accessible to humans. Those expired rodents then begin to decay. Decomposing rodents smell awful, and the stench emitted therefrom is highly undesirable in places where people work and/or congregate. Dead insects do not generally pose a scent problem, but surfaces in the treated air volume have to be cleaned after application of high-concentration ozone in order to remove dead insects. Otherwise, people in that treated space may see those dead insects, and form the incorrect assumption that the treated space is dirty and insect-infested. Further, rotting pests near humans, and/or in the vicinity of food or drink meant for human consumption are a sanitary and health concern. Thus, known pest control systems that utilize ozone at high concentrations are not used in such places.

Thus, there exists a need for a system and method to drive pests out of interior spaces where people work and/or congregate, without the drawbacks of the prior art.

### SUMMARY OF THE INVENTION

An exemplary method for pest deterrence in an interior volume may include moving a portable pest control device into the interior volume; verifying that the interior volume is unoccupied; actuating the said portable pest control device to deliver ozonated air to the interior volume; equilibrating ozone concentration in the interior volume within a selected ozone concentration range for an exposure duration; detecting a completion condition; actuating the portable pest control device to cease delivering ozonated air to the interior volume; and waiting, for an ozone clearance duration, for ozone decomposition.

An exemplary pest control device may include a portable housing; a power bus within the portable housing; a charge controller connected to the power bus; a controller connected to the charge controller; at least one sensor connected to the controller, where at least one sensor is an ozone concentration sensor; a networking module connected to the controller; a fan control switch connected to the controller and to the power bus; a fan connected to the fan control switch; an ozone generator switch connected to the controller and to the power bus; and an ozone generator connected to the ozone generator switch.

In an aspect of the invention, a pest control device generates ozone and directs that ozone into an interior volume of a structure. The pest control devices maintains the concentration of ozone in that interior volume at a level high enough to drive out pests from that interior volume without killing them, across a period of time long enough to cause virtually all of the pests in the interior volume to flee that interior volume.

In an aspect of the invention, a pest control device is portable, such that it can be utilized for pest control in multiple different interior volumes as needed.

In an aspect of the invention, the pest control device may be actuated on and off remotely, such as by an app on a user's smartphone.

In an aspect of the invention, data regarding treatment of an interior volume with the pest control device, such as treatment duration, on and off times, ozone concentration, carbon dioxide concentration, temperature, and humidity is monitored, collected, stored, and analyzed. That monitoring, collection, storage and analysis may be performed at the pest control device, and/or at a server connected to the pest control device.

### BRIEF DESCRIPTION OF THE DRAWINGS

FIG. 1 is a schematic view of a pest deterrent device for pest control.

FIG. 2 is a schematic view of a pest deterrent system utilizing the pest deterrent device of FIG. 1.

FIG. 3 is a flowchart describing a method of use of the pest control device.

The use of the same reference symbols in different figures indicates similar or identical items.

### DETAILED DESCRIPTION OF THE INVENTION

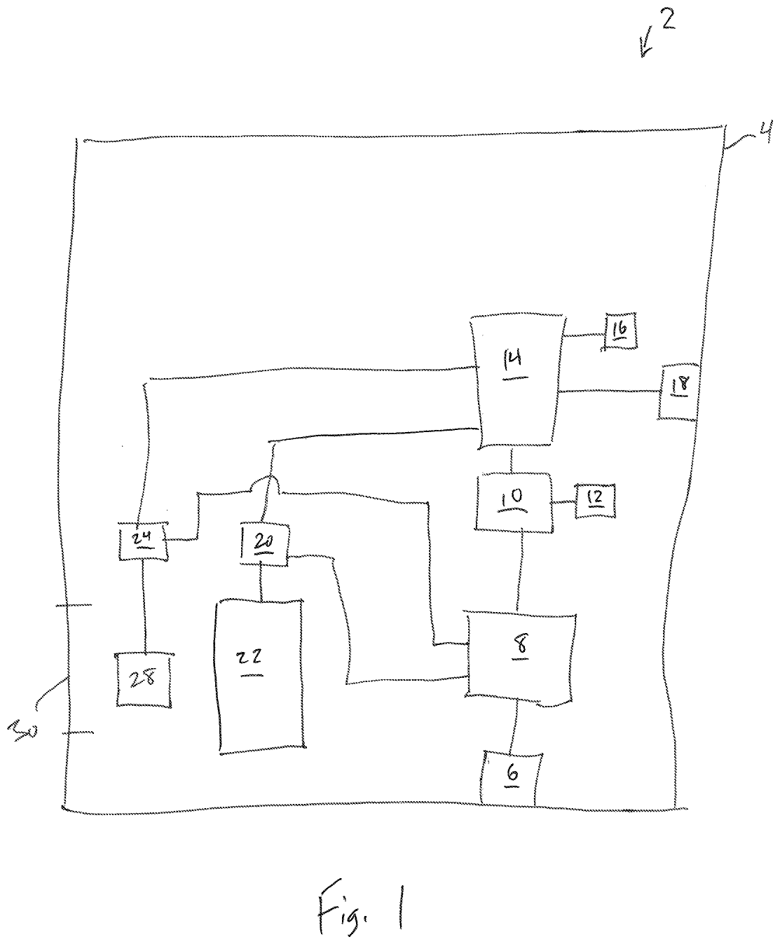

Referring to FIG. 1, an exemplary pest deterrent device 2 is shown. The pest deterrent device 2 may have a housing 4 fabricated from any suitable material, such as aluminum, steel, or plastic. The pest deterrent device 2 is portable. As used in this document, the term “portable” refers to an object that is not permanently affixed to a structure, and that is capable of being moved about by no more than two people. According to some embodiments, the pest deterrent device 2 includes casters connected to the housing 4, and can be rolled from one place to another. According to other embodiments, the pest control device includes one or more exterior handles connected to the housing 4 that can be grasped by one or more users to move the pest deterrent device 2 from one place to another.

A power input 6 may be associated with the housing 4, located inside the housing 4, located outside the housing 4, or otherwise positioned. The power input 6 may be a cord, receptacle, or other structure configured to receive power from a standard power outlet in a structure. The power input 6 may be configured to receive 120 VAC, 220 VAC, three-phase, or any other suitable voltage utilized in the country of use of the pest deterrent device 2. Further, the power input 6 may be configured to receive, or to provide, prongs and a grounding pin consistent with their standard configuration in the country where the pest deterrent device 2 is used. A physical switch may be associated with the power input 6 so that a user can manually power on and power off the pest deterrent device 2. The power input 6 may be connected to a power bus 8, which allows power received from the power input 6 to be split between AC power used to operate some components of the pest deterrent device 2 and power to be stepped down to low-voltage DC power used to control the pest deterrent device 2.

A portion of the power received from the power input 6 at the power bus 8 is directed to a charge controller 10. The charge controller 10 steps down the 120 VAC or other wall power received via the power input 6 and power bus 8 to low voltage DC power, such as but not limited to 3.3V DC power. A backup power source 12 may be connected to the charge controller as well. The backup power source 12 may be a LiPo battery or other battery, and that battery may be rechargeable and kept at a storage level of 80% or higher by the charge controller 10.

The charge controller 10 may be connected to and provide operating power to a controller 14. The controller 14 stores non-transitory instructions for operating the pest deterrent device 2. The controller 14 may be connected to one or more sensors 16 with a data and/or power connection, such as a wire or wires, that provides power to the one or more sensors 16 and receives data from the one or more sensors 16. The one or more sensors 16 may include an ozone concentration sensor, a temperature sensor, a humidity sensor, a flow rate sensor, and a carbon dioxide sensor. One or more additional, or other, types of sensors 16 may be connected to the controller 14. In some embodiments, the controller 14 may be configured to store data received from the one or more sensors. In other embodiments, the controller 14 may direct that data received from one or more sensors to a separate memory, connected to the controller 14 by a physical connection or by a wireless connection.

A networking module 18 may be in electronic and data communication with the controller 14. The networking module 18 may be configured to connect to a computer network such as the internet, whether directly, through an app on the user's smartphone, laptop or other computer, or through a separate standalone device. By way of example and not limitation, the networking module 18 may be configured to connect to one or more wired or wireless networks to transmit and receive data utilizing any suitable protocol. In some embodiments, the wireless network protocols used by the networking module 18 may be one or more of the WI-FI® protocol of the Wi-Fi Alliance of Austin, Texas; the BLUETOOTH® protocol and/or the BLUETOOTH® Low Energy (BLE) protocol of the Bluetooth SIG of Kirkland, Washington; the LoRa® and/or LoRaWAN® protocol of the LoRa Alliance of Fremont, California; the ANT® protocol and/or the ANT+® protocol of Garmin Canada of Cochrane, Alberta; a cellular network such as one utilizing the GSM® protocol and/or LTE® protocol of the European Telecommunications Standards Institute (ETSI), and/or the PCS® standard of the International Telecommunications Union of Geneva, Switzerland; a satellite communication network such as the STARLINK® network of Space Exploration Technologies Corp. of Hawthorne, California; and radio frequency communication. In other embodiments, a wired protocol such as Ethernet may be utilized, and the networking module 18 may contain an Ethernet port for physical attachment by a cable or wire to an Ethernet port in the structure where the pest deterrent device 2 is used.

The power bus 8 may provide AC power to a fan control switch 20, which is connected to a fan 22. The fan control switch 20 may have a data and power connection to the controller 14. As described in greater detail below, the controller 14 is configured to toggle the fan control switch 20 between an off position and an on position. In the off position, the fan control switch 20 prevents power delivery from the power bus 8 to the fan 22. In the on position, the fan control switch 20 provides power from the power bus 8 to the fan 22. Optionally, in some embodiments, the fan control switch 20 is configured to provide variable power to the fan 22 based on instructions received from the controller 14. For example, the controller 14 and fan control switch 20 may be configured to actuate the fan 22 at 0%, 20%, 40%, 60%, 80%, and 100% of full power, rather than simply turning the fan 22 off and on. As another example, the controller 14 and fan control switch 20 are configured to provide more fine-grained control of power delivered to the fan 22. As another example, the controller 14 and fan control switch 20 are configured to provide less fine-grained control of power delivered to the fan 22. As another example, the controller 14 and fan control switch 20 are configured to provide different, fewer, or additional intervals of percentages of full power to the fan 22. As another example, the controller 14 and fan control switch 20 are configured to actuate the fan 22 across a continuous range between 0-100% of full power.

The power bus 8 may provide AC power to an ozone generator switch 24, which is connected to an ozone generator 28. The ozone generator 28 is configured to produce ozone from the oxygen in ambient air. In some embodiments, the ozone generator 28 is a corona discharge ozone generator. In other embodiments, the ozone generator 28 is a UV light ozone generator or other kind of ozone generator. The ozone generator switch 24 may have a data and power connection to the controller 14. As described in greater detail below, the controller 14 is configured to toggle the ozone generator switch 24 between an off position and an on position. In the off position, the ozone generator switch 24 prevents power delivery from the power bus 8 to the ozone generator 28. In the on position, the ozone generator switch 24 provides power from the power bus 8 to the ozone generator 28. Optionally, in some embodiments, the ozone generator switch 24 is configured to provide variable power to the ozone generator 28 based on instructions received from the controller 14. For example, the controller 14 and ozone generator switch 24 may be configured to actuate the ozone generator 28 at 0%, 20%, 40%, 60%, 80%, and 100% of full power, rather than simply turning the ozone generator 28 off and on. As another example, the controller 14 and ozone generator switch 24 are configured to provide more fine-grained control of power delivered to the ozone generator 28. As another example, the controller 14 and ozone generator switch 24 are configured to provide less fine-grained control of power delivered to the ozone generator 28. As another example, the controller 14 and ozone generator switch 24 are configured to provide different, fewer, or additional intervals of percentages of full power to the ozone generator 28. As another example, the controller 14 and ozone generator switch 24 are configured to actuate the ozone generator 28 across a continuous range between 0-100% of full power.

The fan 22 may be located in any suitable part of the housing 4, and is configured to blow outward from the housing 4 through an exit port 30. In some embodiments, the ozone generator 28 may be located in front of the fan 22, such that the fan 22 blows air outwardly from itself onto and into the ozone generator 28, and such that the ozone produced by the ozone generator 28 is blown away from both the fan 22 and the ozone generator 28 into the environment outside the pest deterrent device 2. In other embodiments, the ozone generator 28 may be located behind the fan 22, such that the fan 22 sucks ozonated air into itself from the ozone generator 28, then blows that ozonated air away from both the fan 22 and the ozone generator 28 into the environment outside the pest deterrent device 2. In some embodiments, the fan 22 and ozone generator 28 may be placed in a duct through the housing 4 of the pest deterrent device 2. In other embodiments, the housing 4 may include one or more vents, slots, apertures, or other openings to the environment, and air from the environment is pulled through those one or more vents, slots, apertures, or other openings into and through the fan 22, such that no duct is utilized.

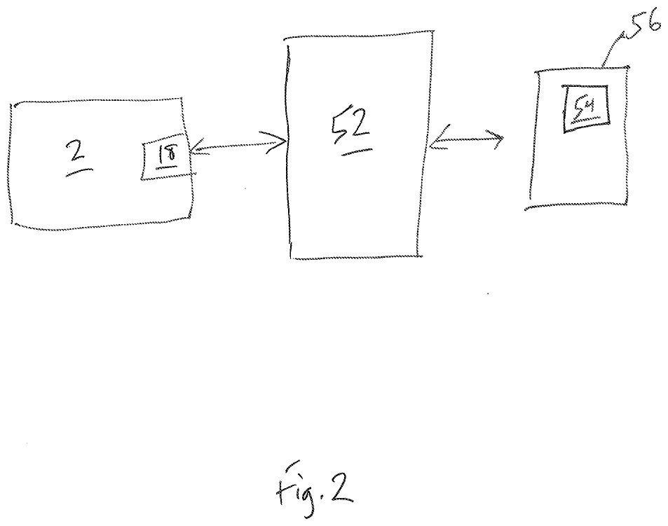

Referring to FIG. 2, the pest deterrent device 2 may be part of a pest deterrent system 50 that includes a server 52, as well as an app 54 that is nontransitory executable software configured to run on a smartphone 56, laptop, tablet, or other device. The pest deterrent device 2 may communicate with the server 52 via the networking module 18, either directly, or indirectly via the Internet or other communications network. As described in greater detail below, the server 52 receives data from the pest deterrent device 2, stores data from the pest deterrent device 2, and/or analyzes data from the pest deterrent device 2. The app 54 may communicate with the server 52 via the networking module 18. The user of the pest deterrent system 50 may utilize a smart phone 56 to receive data from and/or transmit data to the server 52, as described in greater detail below. In some embodiments, the app 54 may be downloadable from an app store in which apps for smart phones 56 are available.

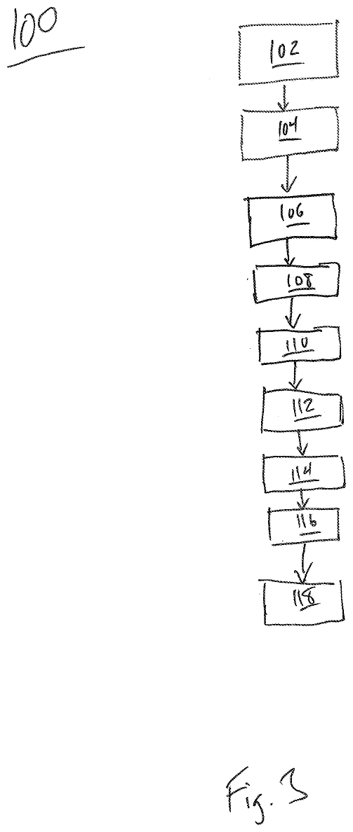

Referring to FIG. 3, according to some embodiments, the pest deterrent device 2 may be operated according to the flowchart 100. At box 102, the pest deterrent device 2 is moved into an interior volume to be treated with ozone for pest control. That interior volume is an enclosed or semi-enclosed space within a structure, and for example may be a bar, restaurant, brewery, winery, distillery, retail store, school, cafeteria, hospital, event center, hotel, commercial kitchen, warehouse, or any other interior volume where pests are an issue. Advantageously, box 102 is performed during a time when the interior volume is unoccupied, or sparsely occupied, particularly when the volume to be treated is relatively small. Alternately, box 102 may be performed at any time, regardless of the number of occupants of the volume. In some embodiments, the pest deterrent device 2 is configured to treat interior volumes of 30,000 cubic feet or less. If treatment of an interior volume of over 30,000 cubic feet is desired, then more than one pest deterrent device 2 may be utilized in that interior volume. If so, the pest deterrent devices 2 are operated according to the flowchart 100. In some embodiments, each pest deterrent device 2 is operated independently of the others. In some embodiments, the pest deterrent devices 2 may be connected to one another by their networking modules 18, and the pest deterrent devices 2 may be operated collectively under the direction of the server 52 and/or a selected one of the pest deterrent devices 2. In some embodiments, the pest deterrent device 2 may be configured to treat interior volumes of greater than 30,000 cubic feet.

At box 104, the pest deterrent device 2 is connected to power. In some embodiments, this is accomplished by connecting the power input 6 of the pest deterrent device 2 to a wall outlet in the interior volume. In other embodiments, this is accomplished by connecting the power input 6 of the pest deterrent device 2 to a generator or other portable power source. In other embodiments, the pest deterrent device 2 may include an on-board battery with a capacity sufficient to power the pest control 2 for a complete cycle of operation; if so, box 104 may be omitted. If there is a manual power switch, the manual power switch may be actuated at this time. Such actuation provides power to the pest deterrent device 2, but does not actuate the ozone generator 28 or fan 22.

Next, at box 106, the user verifies that the interior volume is unoccupied. The user may do so visually, particularly in interior volumes that are relatively small, and where the user is inside or in proximity to the interior volume. If the user is not located in the interior volume, that user may perform verification in other ways. As one example, the user may check that placards and/or barriers to entry to the interior volume have been appropriately placed to warn against entry into the interior volume for a particular span of time, such as while the placards and/or barriers are up, or for a particular duration in time. As another example, the user may follow a lockout procedure, such as used during certain kinds of equipment maintenance. After the user verifies that the interior volume is unoccupied, that user may exit the interior volume and then lock all doors providing access to that interior volume. As another example, the pest deterrent device 2 may include a camera, which allows the user to view the interior volume on the app 54 on the user's smart phone 56. As another example, each employee who routinely works in the interior volume may have the app 54 on their smart phone 56, and the user may send a push notification to each employee's app 54 warning that ozonation of the interior volume is about to begin. Optionally, each employee who receives that push notification may be required to acknowledge that push verification before ozonation is able to begin. Many interior volumes are unoccupied at night, such as stores and restaurants, and verifying that they are unoccupied at night is simple and straightforward.

Next, at box 108, the pest deterrent device 2 is actuated by the user to begin treating the interior volume with ozonated air. Power already has been provided to the controller 14. The user may actuate the pest deterrent device 2 to begin treatment in several ways. In one embodiment, the user may exit the interior volume, and utilize the app 54 on their smart phone 56 to toggle the pest control device to start. In other embodiments, the user may set a timer for actuation of the pest deterrent device 2 using the app 54 on their smart phone. That time may be set immediately before the treatment is to begin, may be set in advance, or may be a recurring timer that always initiates treatment at the same time on the same day (e.g., 11:00 pm Sunday) or on intervals of days (e.g., every fifth day at 1:00 am). In some embodiments, the timer only indicates a start time, and the stop time is determined utilizing data from the sensors 16, as described in greater detail below. In other embodiments, the timer may include a conditional stop time, meaning that the pest deterrent device 2 will cease ozonation of the interior volume at a particular hard stop time, regardless of whether the sensors 16 detect that sufficient ozonation has been performed. This may be important when a shift of workers is scheduled to come to work at a particular time in the interior volume, and the interior volume has to be free of ozone by then. In other embodiments, the timer may include a hard start time and a hard stop time. Such a timer may be useful where the user has experience utilizing the pest deterrent device 2 in a particular interior volume, knows that exposure time is sufficient for pest control, and has tight scheduling needs.

At box 108, once the pest deterrent device 2 receives the command to begin ozonation, via the networking module 18 or via an internal timer, the controller 14 closes the fan control switch 20 and the ozone generator switch 24. The closed fan control switch 20 allows power to flow from the power bus 8 to the fan 22, and the closed ozone generator switch 24 allows power to flow from the power bus 8 to the ozone generator 28. The fan 22 begins to blow air from the interior volume therethrough, and the ozone generator 28 begins to generate oxygen. In some embodiments, the controller 14 may close the fan control switch 20 and start the fan 22 before closing the ozone generator switch 24 to start the ozone generator 28. In this way, buildup of ozone in the housing 4 may be reduced or eliminated. In some embodiments, the controller 14 may close the fan control switch 20 and start the fan 22 after closing the ozone generator switch 24 to start the ozone generator 28. In this way, the initial flow of air away from the fan 22 is more fully ozonated. Ozonated air begins to be pushed out of the housing 4 by the fan 22.

Next, at box 110, the controller 14 equilibrates the interior volume. Ozone concentration in a range of 100-2000 parts per billion (ppb) is optimal for pest control that drives out pests from the interior volume without killing them. Driving pests away from an interior volume without killing those pests is defined in this document as “deterrence.” The one or more sensors 16 monitor the air in the interior volume and transmit data to the controller 18 about ozone concentration; in some embodiments, the one or more sensors also monitor temperature, carbon dioxide concentration, flow rate, and/or humidity. The one or more sensors 16 also may include a timer that monitors exposure time to ozone in the interior volume, where that timer starts when the ozone generator switch 24 is closed.

The controller 14 utilizes data received by the sensor or sensors 16 to control the ozone concentration in the interior volume. When the sensor or sensors 16 indicate that the concentration of ozone in the interior volume is within the range of 100-2000 ppb, normal operation of the pest deterrent device 2 continues. When the sensor or sensors indicate that the concentration of ozone in the interior volume is below 100 ppb, or is approaching a concentration below 100 ppb, the controller 14 may take action. One action is to notify the user of the pest deterrent device 2, such as by initiating a push notification to the app 54 on the user's smart phone 56, via the server 52. The user then may choose to turn off the pest deterrent device 2 remotely, or to allow the pest deterrent device 2 to continue to run.

Another action is to increase the power supplied to the ozone generator 28 or otherwise control the ozone generator 28 to generate more ozone. As described above, the controller 14 and ozone generator switch 24 may be configured to actuate the ozone generator 28 at a percentage of full power, rather than simply turning the ozone generator 28 off and on. For example, during normal operation of the pest deterrent device 2, the controller 14 may be configured to supply 60% or 80% of full power to the ozone generator 28, or a different percentage of power less than 100% of full power, in order to provide headroom for additional power when needed. For example, where the controller 14 applies 80% of full power to the ozone generator 28 during normal operation, and the sensor or sensors 16 report to the controller 14 that the concentration of ozone in the interior volume is below 100 ppb, or is approaching a concentration below 100 ppb, the controller 14 may control the ozone generator switch 28 to increase the power delivered to the ozone generator 28 to a greater percentage of full power in order to increase the amount of ozone delivered to the interior volume.

Another action is to decrease the power supplied to the fan 22 or otherwise control the fan control switch 20 to decrease the flow rate through the fan 22. One of the sensors 16 may be a flow rate sensor that measures the flow rate of air through the fan 22. As described above, the controller 14 and fan control switch 20 may be configured to actuate the fan at a percentage of full power, rather than simply turning the fan 22 and on. For example, during normal operation of the pest deterrent device 2, the controller 14 may be configured to supply 100% of full power to the fan 22, in order to provide headroom for less flow rate when needed. For example, where the controller 14 applies 80% of full power to the fan 22 during normal operation, and the sensor or sensors 16 report to the controller 14 that the concentration of ozone in the interior volume is below 100 ppb, or is approaching a concentration below 100 ppb, the controller 14 may control the fan control switch 20 to decrease the power delivered to the fan 22 to a lesser percentage of full power, such as 60%, in order to decrease the flow rate through the fan 22. Decreasing the flow rate will increase the concentration of ozone in air blown outward from the fan, because a particular quantity of ozone is then mixed with a lesser quantity of air.

Another action is for the controller 14 to both control the ozone generator 28 to generate more ozone and to control the fan 22 to decrease the flow rate of air through the fan.

Still in box 110, when the sensor or sensors indicate that the concentration of ozone in the interior volume is above 2000 ppb, or is approaching a concentration above 2000 ppb, the controller 14 may take action. One action is to notify the user of the pest deterrent device 2, such as by initiating a push notification to the app 54 on the user's smart phone 56, via the server 52. The user then may choose to turn off the pest deterrent device 2 remotely, or to take other action.

Another action is to decrease the power supplied to the ozone generator 28 or otherwise control the ozone generator 28 to generate less ozone. As described above, the controller 14 may be configured to a percentage of full power to the ozone generator 28 less than or equal to 100% of full power, during normal operation. For example, where the controller 14 applies 80% of full power to the ozone generator 28 during normal operation, and the sensor or sensors 16 report to the controller 14 that the concentration of ozone in the interior volume is above 2000 ppb, or is approaching a concentration above 2000 ppb, the controller 14 may control the ozone generator switch 28 to decrease the power delivered to the ozone generator 28 to a lower percentage of full power in order to decrease the amount of ozone delivered to the interior volume.

Another action is to increase the power supplied to the fan 22 or otherwise control the fan control switch 20 to increase the flow rate through the fan 22. One of the sensors 16 may be a flow rate sensor that measures the flow rate of air through the fan 22. As described above, the controller 14 may be configured to supply a percentage of full power to the fan 22 during normal operation. For example, where the controller 14 applies 80% of full power to the fan 22 during normal operation, and the sensor or sensors 16 report to the controller 14 that the concentration of ozone in the interior volume is above 2000 ppb, or is approaching a concentration above 2000 ppb, the controller 14 may control the fan control switch 20 to increase the power delivered to the fan 22 to a higher percentage of full power in order to increase the flow rate through the fan 22. Increasing the flow rate will decrease the concentration of ozone in air blown outward from the fan, because a particular quantity of ozone is then mixed with a greater quantity of air.

Another action is for the controller 14 to both control the ozone generator 28 to generate less ozone and to control the fan 22 to increase the flow rate of air through the fan.

Still in box 110, if the controller 14 detects out-of-spec operation of the pest deterrent device 2 and continues to detect that out-of-spec operation for a length of time that exceeds an acceptable deviation time, the controller 14 may open the ozone generator switch 24, turning off the ozone generator 28 and ceasing production of ozone. The fan control switch 20 may remain closed, especially if the out-of-spec operation is operation at an ozone concentration over 2000 ppb, in order to disperse ozone more effectively from the interior volume. Such automatic shutdown in the event of out-of-spec operation prevents the pest deterrent device 2 from operating at a high enough ozone level that pests are killed in the interior volume, rather than driven out. Further, automatic shutdown in the event of out-of-spec operation at an ozone concentration under 100 ppb prevents the waste of energy of operation at a concentration that does not deter pests. Further, automatic shutdown in the event of out-of-spec operation may prevent further damage to the pest deterrent device 2, if the out-of-spec operation resulted from damage to the pest deterrent device 2 in the first place.

At box 112, concurrently with box 110, the controller 14 causes data received from the sensors 16 to be stored. The controller 14 may cause that data received from the sensors 16 to be stored locally on the pest deterrent device 2, and/or may cause data receive from the sensors 16 to be transmitted to the server 52 via the networking module 18, where the server 52 causes that data to be stored at the server 52 itself or at another storage location accessible to the server 52. Also at box 112, the controller 14 causes data received from the sensors 16 to be transmitted to the app 54 of the user's smart phone 56, by transmitting that data through the networking module 18 to the server 5. In some embodiments, the server 52 may push that data to the app 54 of the user's smart phone 56 in a steady stream. In some embodiments, the server 52 may push that data to the app 54 of the user's smart phone 56 only when at least one condition measured by the sensors 16 is out of spec. In some embodiments, the user may utilize the app 54 on the user's smart phone to send a pull notification to the server 52, after which the server 52 transmits data received from the sensors 16 to the app 54 on the user's smart phone 56. The user may request, and/or receive, near real-time data from the pest deterrent device 2 via the server 52. In some embodiments, the user may request, and/or receive, historical data from the pest deterrent device 2 that is stored on the pest deterrent device 2, at or in association with the server 52, and/or on the user's smart phone 56 in association with the app 54. Also at box 110, the server 52 may analyze the stored data. That analysis may be with regard to the particular treatment ongoing in box 110, may be in the aggregate using data from other operations of the portable pest deterrent device 2 in the same interior volume or different interior volumes, may be in the aggregate using data from operations of other, different portable pest deterrent devices 2, or may be any other analysis that is desirable for the user.

At box 114, the pest deterrent device 2 continues ozonation until a completion condition is met. Ozone concentration of 100-2000 ppb applied to an interior volume of 30,000 cubic feet or less for an exposure duration of 3-18 hours has been found to deter pests, driving them out of the interior volume rather than killing them within the interior volume, which as described above is undesirable for many reasons. The ranges of ozone concentration and exposure duration arise primarily from the type of pest that is sought to be deterred. Some interior volumes to be treated may have a particular issue with or interest in deterring rodents; others may have a particular issue with or interest in deterring birds, insects, or other pests. The particular ozone concentration and exposure duration for treating each particular type of pest is different. Because the ozone concentration used to treat the interior volume is different for different pests, the sensors 16 are particularly important for the pest deterrent device 2, the pest deterrent system 50, and the flowchart 100. Otherwise, without measuring and controlling ozone concentration to deter specific pests, the pest deterrent device 2 may not actually deter the pests sought to be deterred. Worse, without measuring and controlling ozone concentration to deter specific pests, the pest deterrent device 2 may kill rather than deter pests from the interior volume-causing the issues with pest carcasses and decay that occur with known devices, which the pest deterrent device 2, the pest deterrent system 50, and the flowchart 100 have been created to overcome. Further, operation for an exposure duration at an unintentionally high ozone concentration may cause dangerous conditions in and near the interior volume, and/or harm to the pest deterrent device 2. For this reason as well, monitoring ozone concentration with one or more of the sensors 16 is important for ensuring the health not only of the pest deterrent device 2, but also for humans in the vicinity of the interior volume.

The ranges of ozone concentration and duration also may be related to differences in different interior volumes and their individual characteristics, among other factors. As one example, an interior volume with vents and multiple spaces for leakage of ozone may require a higher ozone concentration and/or higher exposure duration to meet the completion condition. As another example, a smaller interior volume may require a lower ozone concentration and/or a lower exposure duration to meet the completion condition. These differences in interior volume and leakage are important primarily insofar as they relate to the specific ozone concentration and exposure duration required to deter specific pests, as set forth in the paragraph above. Particular interior volumes may require adjustment of initial ozone concentration and exposure duration parameters based on experience utilizing the pest deterrent device 2 in those volumes.

The completion condition may be simple exposure duration. As described above, one of the sensors 16 may be a time sensor—that is, a timer that monitors exposure time to ozone in the interior volume, where that timer starts when the ozone generator switch 24 is closed. In other embodiments, the completion condition may be an exposure duration, where any time the pest deterrent device 2 is operating out-of-spec is not counted toward the completion condition. For example, if the exposure duration for completion is 5 hours, and the pest deterrent device 2 operated out of spec for 10 minutes, the completion condition would not be met until the pest deterrent device 2 operated for 5 hours and 10 minutes. In other embodiments, the completion condition may be a total ozone amount delivered to the interior volume regardless of exposure duration. That is, integrating the curve of ozone concentration over time results in a total ozone amount delivered to the interior volume; the controller 14 may perform this calculation to determine when the completion condition is met.

Still in box 112, when the completion condition is met, the controller 14 opens the ozone generator switch 24, shutting off power to the ozone generator 28. The ozonation process is complete.

Next, in box 116, the sensor or sensors 16 associated with the pest control device 2 monitor the degradation of ozone in the interior volume to determine when that concentration of ozone has been reduced to a concentration low enough that humans are safe to enter the interior volume. The time taken for ozone to degrade to a safe concentration may be referred to as the ozone degradation interval. The controller 14 may leave the fan control switch 20 in the closed state, continuing to deliver power to the fan 22 so that the fan 22 can continue moving air in the interior volume to assist with degrading the ozone in the interior volume. In some embodiments, data from the sensor or sensors 16 may be pushed to the app 54 on a user's smart phone 56 via the server 52 in real time or near real time, so that the user is aware of the concentration of ozone in that interior volume at all times. In some embodiments, the user may pull data from the sensor or sensor 16 to the app 54 on the user's smart phone 56 via the server 52.

The National Institute for Occupational Safety and Health (NIOSH) sets forth permissible exposure limits (PELs) for humans for certain hazardous chemicals, like ozone. Those PELs include time-weighted average (TWA) concentrations for up to a 10-hour workday during a 40-hour workweek, and a short-term exposure limit (STEL), which is a 15-minute TWA exposure that should not be exceeded at any time during a workday. The NIOSH PEL for airborne ozone exposure is 0.1 parts per million (ppm), which equates to 100 ppb. (NIOSH Pocket Guide to Chemical Hazards, https://www.cdc.gov/niosh/npg/npgd0476.html). The TWA for airborne ozone exposure is also 0.1 parts per million (ppm), which equates to 100 ppb. (Id.). The STEL for ozone is 0.3 ppm, which equates to 300 ppb. (NIOSH Table of IDLH Values, accessed at https://www.cdc.gov/niosh/idlh/10028156.html). Importantly, NIOSH identifies concentrations of hazardous chemicals that are immediately dangerous to life or health (IDLH). The IDLH value of ozone is 5 ppm, which equates to 5000 ppb. (Id.). Individual states and other countries may have stricter limits for ozone exposure.

The controller 14 may be programmed with the maximum allowable ozone concentration (PEL, TWA, STEL and/or other limits) for humans in the location of use of the pest deterrent device 2. When the sensor or sensors 16 detect an ozone concentration in the interior volume that is less than or equal to the maximum allowable ozone concentration, the controller 14 may open the fan control switch 20 and turn off the fan 22, if the fan 22 is running. Allowing the fan 22 to run during the ozone degradation interval not only helps the ozone to disperse and degrade, but also mixes and homogenizes the air in the interior volume so that the sensor or sensors 16 obtain a more accurate reading of ozone concentration in the interior volume as a whole, rather than sensing only a localized portion of the interior volume, which may not be representative of the ozone concentration in the interior volume as a whole. Additionally, or instead, the controller 14 may transmit a push notification to the app 54 on the user's smart phone 56 notifying the user that the ozone concentration in the interior volume has reached a level safe for human exposure. In general, in most interior volumes, degradation of ozone to a safe concentration takes 30 minutes to three hours, depending on ventilation, temperature and other factors. In some cases, that time may be less than 30 minutes or longer than three hours. Monitoring the ozone concentration, rather than relying on an arbitrary rule-of-thumb to guess when the ozone concentration might be safe, ensures that the interior volume is safe for humans to enter and stay in that space after the pest deterrent device 2 has ozonated the interior volume and deterred pests from within it.

Next, at box 118, the pest deterrence process is complete. One or more people may enter the interior volume, and remove the pest deterrent device 2.

As used in this document, and as customarily used in the art, the word “substantially” and similar terms of approximation refer to normal variations expected in manufacturing and communications; for example, from normal variations in network and communications properties, and in the measurement of such variations.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the claims.

What is claimed is:

1. A method for pest deterrence in an interior volume, comprising:
moving a portable pest deterrent device into the interior volume;
verifying that the interior volume is unoccupied;
actuating said portable pest deterrent device to deliver ozonated air to the interior volume;
equilibrating ozone concentration in the interior volume within a selected ozone concentration range for an exposure duration;
detecting a completion condition;
actuating said portable pest deterrent device to cease delivering ozonated air to the interior volume; and
waiting, for an ozone clearance duration, for ozone decomposition.

2. The method of claim 1, wherein said ozone concentration range is 100-2000 ppb.

3. The method of claim 1, wherein said exposure duration is 3-18 hours.

4. The method of claim 1, wherein said ozone clearance duration is 30 minutes-3 hours.

5. The method of claim 1, wherein said waiting further comprises measuring ozone concentration in the interior volume, and wherein said ozone clearance duration is determined by said measuring.

6. The method of claim 1, wherein said actuating said portable pest deterrent device to deliver ozonated air to the interior volume comprises actuating an ozone generator to generate ozone, and blowing the ozone into the interior volume with a fan.

7. The method of claim 6, wherein said equilibrating further comprises sensing an ozone concentration in the interior volume, and upon sensing an ozone concentration higher than the ozone concentration range, performing at least one of (a) increasing power to said fan to increase flow rate, and (b) decreasing power to said ozone generator to decrease ozone production.

8. The method of claim 6, wherein said equilibrating further comprises sensing an ozone concentration in the interior volume, and upon sensing an ozone concentration lower than the ozone concentration range, performing at least one of (a) decreasing power to said fan to decrease flow rate, and (b) increasing power to said ozone generator to increase ozone production.

9. The method of claim 6, wherein said waiting further comprises continuing to actuate said fan.

10. The method of claim 1, wherein said completion condition is exposure duration.

11. The method of claim 1, further comprising connecting said portable pest deterrent device to a server, and connecting said server to a smart phone on which executable software associated with said portable pest deterrent device is configured to run.

12. The method of claim 1, wherein said equilibrating further comprises transmitting data related to said equilibrating to said server; further comprising storing said data at said server.

13. The method of claim 12, further comprising analyzing said data at said server after said storing.

14. The method of claim 1, wherein said actuating said portable pest deterrent device to deliver ozonated air to the interior volume is performed by said app.

15. The method of claim 1, further comprising sensing at least one of carbon dioxide concentration, temperature, and humidity during said equilibrating.

16. The method of claim 1, further comprising moving at least one additional portable pest deterrent device into the interior volume.

17. A pest deterrent device, comprising:
a portable housing;
a power bus within said portable housing;
a charge controller connected to said power bus;
a controller connected to said charge controller;
at least one sensor connected to said controller, wherein at least one said sensor is an ozone concentration sensor;
a networking module connected to said controller;
a fan control switch connected to said controller and to said power bus;
a fan connected to said fan control switch;
an ozone generator switch connected to said controller and to said power bus; and
an ozone generator connected to said ozone generator switch.

18. A pest deterrent system, comprising:
the pest deterrent device of claim 15;
a server in data communication with said pest control device;
a device in communication with said server, said device comprising at least one processor and a non-transitory computer-readable storage medium; and
an app comprising instructions that configure said at least one processor.

19. The pest deterrent system of claim 18, wherein said instructions comprise instructions to:
actuate said portable pest deterrent device to deliver ozonated air to the interior volume;
receive information from said portable pest deterrent device regarding equilibration of ozone concentration in the interior volume within a selected ozone concentration range for an exposure duration;
receive information from said portable pest deterrent device regarding its detection of a completion condition; and
actuate said portable pest deterrent device to cease delivering ozonated air to the interior volume.
